(19) United States
(12) Patent Application Publication
(10) Pub. No.: US 2025/0254025 A1
(43) Pub. Date: Aug. 7, 2025

(54) SYSTEMS AND METHODS OF LAYERING SECURITY FOR CELLULAR-ENABLED BLOOD OXYGEN SATURATION DATA TRANSMISSION

(71) Applicant: Smart Meter Corporation, Tampa, FL (US)

(72) Inventor: Benjamin Atkin, Bergenfield, NJ (US)

(73) Assignee: Smart Meter Corporation, Tampa, FL (US)

(21) Appl. No.: 18/985,958

(22) Filed: Dec. 18, 2024

Related U.S. Application Data
(63) Continuation-in-part of application No. 17/517,841, filed on Nov. 3, 2021.
(60) Provisional application No. 63/115,935, filed on Nov. 19, 2020.

(51) Int. Cl. H04L 9/06; A61B 5/00; A61B 5/1455; H04L 12/46; H04W 12/033; H04W 12/088

(52) U.S. Cl. CPC H04L 9/0618; A61B 5/0004; A61B 5/14551; H04L 9/0643; H04L 12/4641; H04W 12/033; H04W 12/088

(57) ABSTRACT

A system for improving blood oxygen saturation data transmission security comprising: a pulse oximeter; a wireless network connected to the pulse oximeter; a private network connected to the wireless network via an IPsec VPN tunnel; one or more computer processors; and a memory storing machine executable instructions, that when executed, cause the system to: collect, blood oxygen saturation data from a patient; encrypt, the blood oxygen saturation data with a shared secret, creating encrypted blood oxygen saturation data; generate, a first hash using a signing algorithm; transmit, the encrypted blood oxygen saturation data from the pulse oximeter to the private network; generate, a second hash; compare, the first hash to the second hash; decrypt, the encrypted blood oxygen saturation data upon a match of the first and second hash, creating verified blood oxygen saturation data; and transmit, the verified blood oxygen saturation data to a target recipient.

### CROSS REFERENCE TO RELATED APPLICATIONS

The present application claims the benefit of U.S. patent application Ser. No. 17/517,841 for PULSE OXIMETER WITH CELLULAR COMMUNICATION CAPABILITY, filed Nov. 3, 2021, and U.S. Patent Application No. 63/115,935 for PULSE OXIMETER WITH CELLULAR COMMUNICATION CAPABILITY, filed Nov. 19, 2020, the entire contents of which are incorporated herein by reference.

### FIELD OF THE INVENTION

The present disclosure is directed to a pulse oximeter. More specifically, the present disclosure is directed to systems and methods of layering security for cellular-enabled blood oxygen saturation data transmission.

### INTRODUCTION

Blood oxygen saturation (also known as “SpO2”), is a critical measure of how well oxygen is being distributed throughout the body via the bloodstream. It represents the percentage of hemoglobin in the blood that is saturated with oxygen. Monitoring SpO2 levels is essential because it provides valuable insights into respiratory function and overall health, particularly in individuals with chronic conditions, during acute illness, or under anesthesia. Pulse oximeters are commonly used to measure SpO2 levels.

One of the primary reasons for monitoring blood oxygen saturation is the early detection of hypoxemia, a condition characterized by abnormally low levels of oxygen in the blood. Hypoxemia can be life-threatening if not promptly addressed, as it indicates that the body's tissues and organs may not be receiving adequate oxygen to function properly. A pulse oximeter allows for real-time monitoring of SpO2 levels, enabling healthcare providers and patients to detect hypoxemia early and take appropriate action, such as administering supplemental oxygen or adjusting medical treatments. This early detection is particularly important for individuals with respiratory conditions like chronic obstructive pulmonary disease (COPD), asthma, or pneumonia, where oxygen levels can fluctuate rapidly.

For individuals with chronic respiratory conditions, regular monitoring of blood oxygen saturation is crucial in managing their health. Conditions such as COPD, pulmonary fibrosis, and sleep apnea can lead to chronic hypoxemia, which, if left unmanaged, can cause complications such as heart problems and decreased quality of life. By using a pulse oximeter, patients and healthcare providers can track oxygen levels over time, assess the effectiveness of treatments, and make informed decisions about the need for interventions such as oxygen therapy. Continuous monitoring can also help to prevent acute exacerbations, allowing for early intervention and reducing the likelihood of hospitalization.

Accordingly, it would be desirable to provide systems and methods for better safeguarding blood oxygen saturation data. Furthermore, it would also be desirable to provide systems and methods utilizing multiple layers of protection for transmitting blood oxygen saturation data to patients and/or healthcare providers. Therefore, it would be beneficial to provide the systems and methods of layering security for cellular-enabled blood oxygen saturation data transmission described within the present disclosure.

### SUMMARY

Bearing in mind the problems and deficiencies of the prior art, it is therefore an object of the present disclosure to provide a process and system for social interaction and community building.

Aspects of the present disclosure relate to a system for improving the security of cellular-enabled blood oxygen saturation data transmission by layering security. The system being comprised of: a pulse oximeter; a wireless network connected to the pulse oximeter; a private network connected to the wireless network via a persistent and fully redundant Internet Protocol Security (IPsec) Virtual Private Network (VPN) tunnel; one or more computer processors; and a memory having stored therein machine executable instructions, that when executed by the one or more processors, cause the system to: collect, via the pulse oximeter, blood oxygen saturation data from a patient; encrypt, via the pulse oximeter, the blood oxygen saturation data with a shared secret, wherein encrypting the blood oxygen saturation data creates encrypted blood oxygen saturation data; generate, via the pulse oximeter, a first hash using a signing algorithm; transmit, via the persistent and fully redundant IPsec VPN tunnel, the encrypted blood oxygen saturation data from the pulse oximeter to the private network; generate, via the private network, a second hash; compare, via the one or more computer processors, the first hash to the second hash; decrypt, via the one or more computer processors, the encrypted blood oxygen saturation data upon a match of the first and second hash, wherein decrypting the encrypted blood oxygen saturation data creates verified blood oxygen saturation data; and transmit, via the one or more computer processors, the verified blood oxygen saturation data to a target recipient.

Aspects of the present disclosure relate to a system wherein the shared secret is a symmetric-key algorithm comprising: a key; and a symmetric block cipher. In an embodiment, the key is comprised of at least one of a 128-bit key, a 256-bit key, a 576-bit key, and a 2040-bit key. In a further embodiment, the symmetric block cipher is comprised of at least one of an Advanced Encryption Standard (AES) block cipher, a Blowfish block cipher, a CAST-256 block cipher, a GOST block cipher, an International Data Encryption Algorithm (IDEA) block cipher, a Rivest Cipher 6 (RC-6) block cipher, a Serpent block cipher, and a Twofish block cipher. In yet a further embodiment, the persistent and fully redundant IPsec VPN tunnel leverages the symmetric-key algorithm to encrypt the encrypted blood oxygen saturation data while travelling through the persistent and fully redundant IPsec VPN tunnel.

Aspects of the present disclosure relate to a system wherein the pulse oximeter connects to the wireless network via an APN.

Aspects of the present disclosure relate to a system wherein the persistent and fully redundant IPsec VPN tunnel is further comprised of TLS.

Aspects of the present disclosure relate to a system wherein the verified blood oxygen saturation data is transmitted to one or more client devices of the target recipient.

Aspects of the present disclosure relate to a system wherein the signing algorithm is comprised of at least one of Rivest-Shamir-Adleman (RSA) algorithms, EIGamal signature scheme, Digital Signing Algorithm (DSA), and Elliptical Curve Digital Signature Algorithm (ECDSA).

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates an embodiment of an environment in which the present disclosure may be practiced.

FIG. 2 illustrates an embodiment of a block diagram of an electronic device.

FIG. 3 illustrates an embodiment of a schematic diagram of a traditional pulse oximetry system.

FIG. 4 illustrate s a block diagram of a traditional pulse oximetry system.

FIG. 5 illustrates a schematic diagram of a pulse oximetry system of the present disclosure.

FIG. 6 illustrates an embodiment of a system of layering security for cellular-enabled blood oxygen saturation data transmission.

FIG. 7 illustrates an embodiment of a method of layering security for cellular-enabled enabled blood oxygen saturation data transmission.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

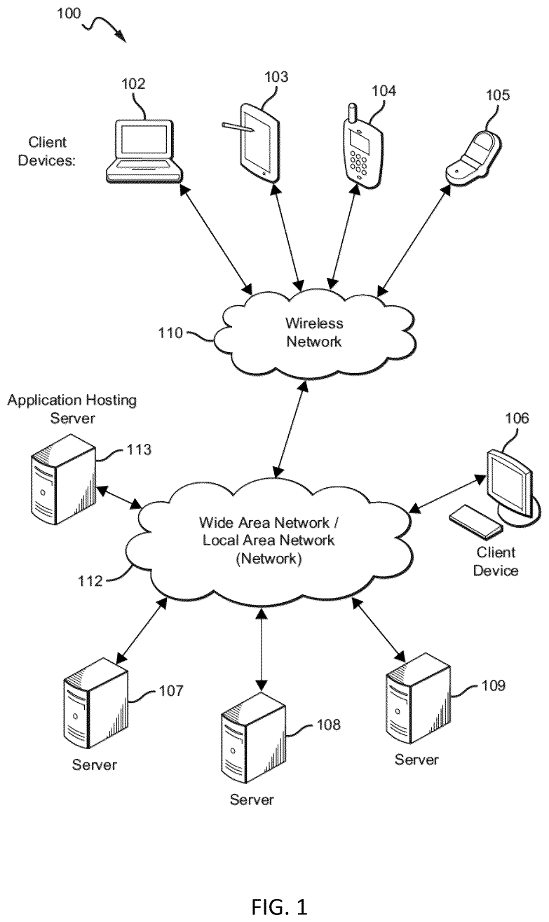

FIG. 1 illustrates components of one embodiment of an environment in which the present disclosure may be practiced. Not all of the components may be required to practice the present disclosure, and variations in the arrangement and type of the components may be made without departing from the spirit or scope of the present disclosure. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. The client devices 102-106 may include, for example, at least one of desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

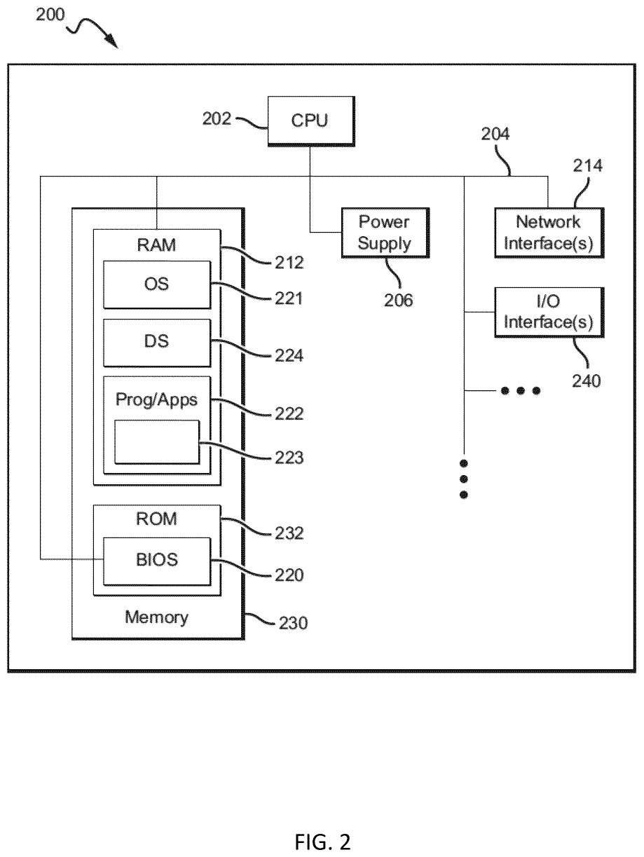

FIG. 2 illustrates a block diagram of an electronic device 200 that can implement one or more aspects of an apparatus, system, and/or method for layering security of cellular-enabled telemetered data (the “Engine”) according to one embodiment of the present disclosure. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software, and other tools necessary to implement or facilitate methods and systems according to embodiments of the present disclosure. The elements may exist on a single computer or be distributed among multiple computers, servers, devices, or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can case processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system, and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone, or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending, and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

As described herein, pulse oximetry is a non-invasive method for monitoring a person's oxygen saturation. Oxygen saturation is the fraction of oxygen-saturated hemoglobin relative to total hemoglobin in the blood. The human body requires and regulates a precise and specific balance of oxygen in the blood. Normal arterial blood oxygen saturation levels in humans are between 95 percent to 100 percent. If the level is below 90 percent, it is considered low and is called hypoxemia. Arterial blood oxygen levels below 80 percent may compromise organ function, such as the brain and heart, and should be promptly addressed. Continued low oxygen levels may lead to respiratory or cardiac arrest. Oxygen therapy may be used to assist in raising blood oxygen levels.

Pulse oximetry is the current standard of care for the continuous monitoring of arterial oxygen saturation (SpO2). Pulse oximeters provide instantaneous in vivo measurements of arterial oxygenation, and thereby provide early warning of arterial hypoxemia. A typical pulse oximeter comprises a computerized measuring unit and a probe attached to the patient, typically to his or her finger. The probe includes a light source for sending an optical signal through the tissue and a photodetector for receiving the signal after transmission through the tissue. On the basis of the transmitted and received signals, light absorption by the tissue can be determined.

During each cardiac cycle, light absorption by the tissue varies cyclically. During the diastolic phase, absorption is caused by venous blood, tissue, bone, and pigments, whereas during the systolic phase, there is an increase in absorption, which is caused by the influx of arterial blood into the tissue. Pulse oximeters focus the measurement on this arterial blood portion by determining the difference between the peak absorption during the systolic phase and the constant absorption during the diastolic phase. As such, pulse oximetry assumes that the pulsatile component of the absorption is due to arterial blood only.

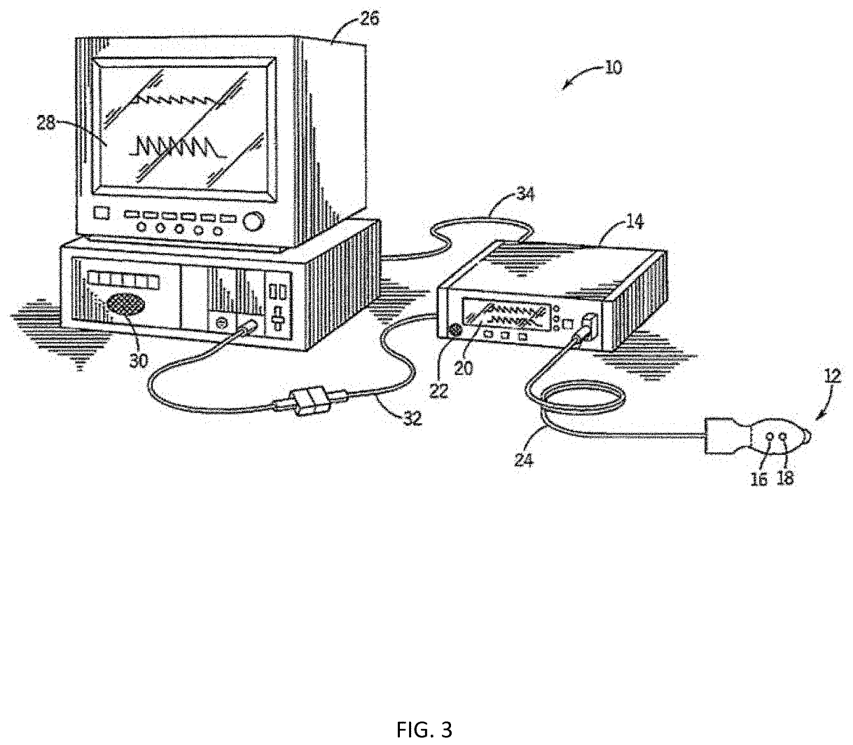

FIG. 3 is a perspective view of an embodiment of a traditional pulse oximetry system 10. The pulse oximetry system 10 of FIG. 3 includes numerous components, such as: a sensor 12 (e.g., a probe) and/or a pulse oximetry monitor 14, among others not explicitly depicted herein. It should be appreciated that the pulse oximetry system 10 may have multiple user functionality and may be beneficial for those individuals needing to consistently track health parameters, such as one's oxygen saturation.

Moreover, the sensor 12 includes an emitter 16 for emitting light at one or more wavelengths into a patient's tissue. The sensor 12 also includes a detector 18 that may detect the light originating from the emitter 16 emanating from the patient's tissue after passing through said tissue. The emitter 16 and detector 18 may be disposed upon opposite sides of a user's finger, which is received by the sensor 12, in which case the light that is emanating from the tissue has passed completely through the user's finger.

The sensor 12 may be connected to and draw power from the monitor 14. Alternatively, the sensor 12 may be wirelessly connected to the monitor 14 and include its own battery or power supply (not shown). The monitor 14 may be configured to calculate physiological parameters based on data received from the sensor 12 relating to light emission and detection.

Further, the monitor 14 includes a display 20 configured to display the physiological parameters and/or other data. In the embodiment shown, the monitor 14 also includes a speaker 22 to provide an audible alarm in the event that the patient's physiological parameters are not within a predetermined range, as defined based on patient characteristics. As depicted, the sensor 12 is communicatively coupled to the monitor 14 via a first cable 24 or other similar means. However, in other embodiments a wireless transmission device (not shown) or the like may be utilized instead of or in addition to the first cable 24.

In the illustrated embodiment of FIG. 3, the pulse oximetry system 10 also includes a multi-parameter patient monitor 26. The multi-parameter patient monitor 26 may be configured to calculate physiological parameters and to provide a central display 28 for information from the monitor 14 and from other medical monitoring devices or systems (not shown). For example, the multiparameter patient monitor 26 may be configured to display a patient's oxygen saturation reading generated by the pulse oximetry monitor 14, pulse rate information from the monitor 14, and a blood pressure reading from a blood pressure monitor (not shown) on the display 28. Additionally, the multi-parameter patient monitor 26 may emit a visible or audible alarm via the display 28 and/or a speaker 30 if the patient's physiological characteristics are found to be outside of the predetermined range defined as “normal.”

The monitor 14 may be communicatively coupled to the multi-parameter patient monitor 26 via a second cable 32 or a third cable 34 coupled to a sensor input port or a digital communications port, respectively. In addition, the monitor 14 and/or the multi-parameter patient monitor 26 may be connected to a network to enable the sharing of information with servers or other workstations (not shown). The monitor 14 may be powered by a battery (not shown) or by a power source, such as a wall outlet.

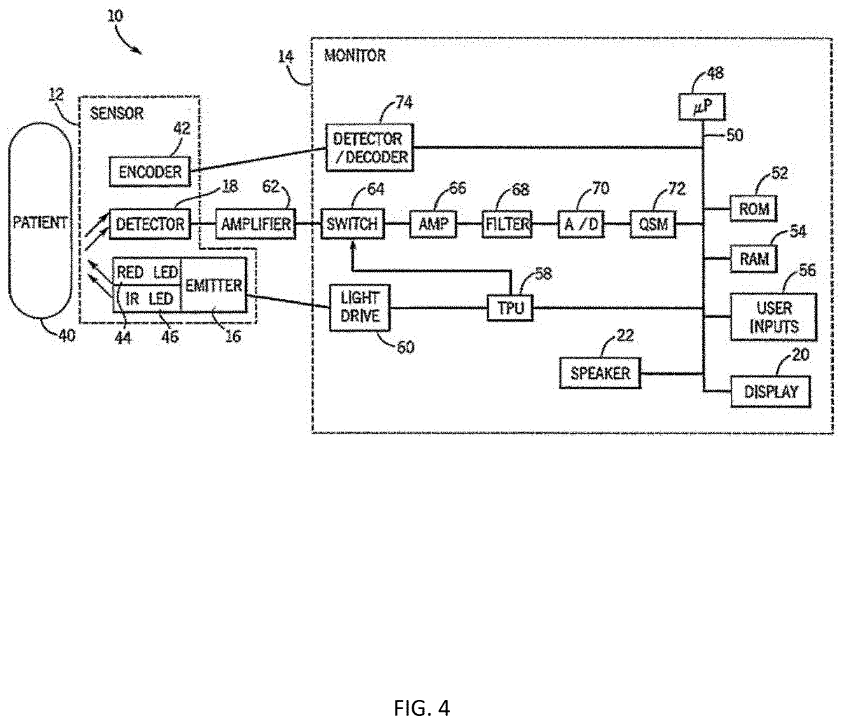

FIG. 4 is a block diagram of the traditional pulse oximetry system 10 of FIG. 3 coupled to a patient 40 in accordance with present embodiments. Specifically, the sensor 12 includes the emitter 16, the detector 18, and an encoder 42. The emitter 16 is configured to emit at least two wavelengths of light, e.g., RED and IR, into the patient's tissue 40. As such, the emitter 16 may include a RED light source (such as a RED LED 44) and an IR light source (such as an IR LED 46) for emitting light into the patient's tissue 40 at the wavelengths used to calculate the patient's physiological parameters (i.e., blood oxygen saturation and pulse rate). In some examples, the wavelength of the RED LED 44 may be between about 600 nm and about 700 nm and the wavelength of the IR LED 46 may be between about 800 nm and about 1000 nm. It should be appreciated that these ranges are provided for illustrative purposes only.

Moreover, it should be appreciated that the quantity of the LED's is not limited to two and other quantities are contemplated herein. Alternative light sources may be used in other embodiments. For example, a single wide-spectrum light source may be used and the detector 18 may be configured to detect light only at certain wavelengths.

It should be understood that, as used herein the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet or X-ray spectra, and that any suitable wavelength of light may be appropriate for use with the present techniques.

In an embodiment, the detector 18 may be configured to detect the intensity of light at the RED and IR wavelengths. In operation, the light enters the detector 18 after passing through the patient's tissue 40. The detector 18 converts the intensity of the received light into an electrical signal. The light intensity is directly related to the absorbance and/or reflectance of light in the patients' tissue 40. As such, when more light at a certain wavelength is absorbed or reflected, less light of that wavelength is received from the tissue by the detector 18. After converting the received light to an electrical signal, the detector 18 sends the signal to the monitor 14, where the physiological parameters may be calculated based on the absorption of the RED and IR wavelengths in the patient's tissue 40.

The encoder 42 may contain information about the sensor 12, such as an identification of what type of sensor it is (e.g., whether the sensor is intended for placement on a forehead or the finger of the user) and the wavelengths of light emitted by the emitter 16. This information may be used by the monitor 14 to select appropriate algorithms, lookup tables and/or calibration coefficients stored in the monitor 14 for calculating the patient's physiological parameters.

In addition, the encoder 42 may contain information specific to the patient 40. Such information may include: the patient's age, the patient's gender, the patient's weight, and/or the patient's diagnosis, among other information. This information may allow the monitor 14 to determine patient-specific threshold ranges in which the patient's physiological parameter measurements should fall and to enable or disable additional physiological parameter algorithms. The encoder 42 may, for instance, be a coded resistor that stores values corresponding to the type of the sensor 12, the wavelengths of light emitted by the emitter 16, and/or the patient's characteristics. These coded values may be communicated to the monitor 14, which determines how to calculate the patient's physiological parameters and alarm threshold ranges.

In another embodiment, the encoder 42 may include a memory that may store information, which is then communicated to the monitor 14. Such information may include: the type of the sensor 12, the wavelengths of light emitted by the emitter 16, the proper calibration coefficients and/or algorithms to be used for calculating the patient's physiological parameters and/or alarm threshold values, the patient characteristics to be used for calculating the alarm threshold values, and the patient-specific threshold values to be used for monitoring the physiological parameters.

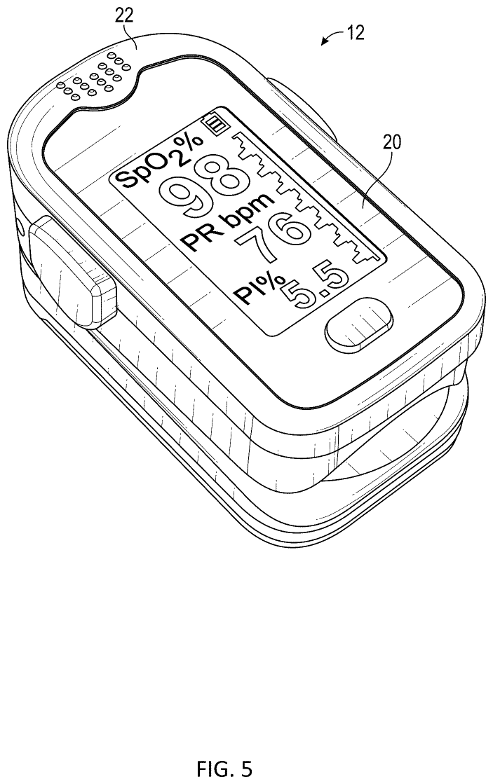

Signals from the detector 18 and the encoder 42 may be transmitted to the monitor 14. As shown in FIG. 4, the monitor 14 includes a general-purpose microprocessor 48 connected to an internal bus 50. The microprocessor 48 is adapted to execute software, which may include an operating system and one or more applications (such as a voice activation component 76 of FIG. 5), as part of performing the functions described herein. A read-only memory (ROM) 52, a random access memory (RAM) 54, user inputs 56, the display 20, and the speaker 22 are also connected to the interface bus 50.

The RAM 54 and ROM 52 are portrayed for illustrative purposes only. Any computer-readable media may be used in the system for data storage. Computer-readable media are capable of storing information that can be interpreted by the microprocessor 48. This information may be data or may take the form of computer-executable instructions, such as software applications, that cause the microprocessor to perform certain functions and/or computer-implemented methods. Depending on the embodiment, such computer-readable media may comprise computer storage media and communication media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

As shown in FIG. 4, a time processing unit (TPU) 58 provides timing control signals to a light drive circuitry 60, which controls when the emitter 16 is illuminated and multiplexed timing for the RED LED 44 and the IR LED 46. The TPU 58 also controls the gating-in of signals from detector 18 through an amplifier 62 and a switching circuit 64, as shown in FIG. 4. These signals are sampled at the proper time, depending upon which light source is illuminated. The received signal from the detector 18 may be passed through an amplifier 66, a low pass filter 68, and an analog-to-digital (AID) converter 70. The digital data may then be stored in a queued serial module (QSM) 72 (or buffer) for later downloading to the RAM 54 as the QSM 72 fills up. In one embodiment, there may be multiple separate parallel paths having the amplifier 66, the filter 68, and the A/D converter 70 for multiple light wavelengths or spectra received.

The microprocessor 48 may determine the patient's physiological parameters, such as SpO2 reading and the pulse rate, using various algorithms and/or look-up tables based on the value of the received signals corresponding to the light received by the detector 18. Signals corresponding to information about the patient 40, and particularly about the intensity of light emanating from a patient's tissue over time, may be transmitted from the encoder 42 to a decoder 74. These signals may include, for example, encoded information relating to patient characteristics. The decoder 74 may translate these signals to enable the microprocessor to determine the thresholds based on algorithms or look-up tables stored in the ROM 52.

The encoder 42 may also contain the patient-specific alarm thresholds if the alarm values are determined on a workstation separate from the monitor 14. The user inputs 56 may also be used to enter information about the patient, such the patient's age, the patient's gender, the patient's height, the patient's weight, medications the patient is taking, treatments the patient is engaging in, and/or the patient's diagnosis, among others. In some examples, the display 20 may exhibit a list of values that may generally apply to the patient, such as, for example, age ranges or medication families, which the user may select using the user inputs 56. The microprocessor 48 may then determine the proper thresholds using the user input data and algorithms stored in the ROM 52. The patient-specific thresholds may be stored on the RAM 54 for comparison to measured physiological characteristics. The ROM 52 and the RAM 54 may also store information for use in selection of a power consumption mode based on the data generated by the sensor 12 and/or monitor 14.

FIG. 5 depicts a schematic diagram of the pulse oximetry system 10 of the present invention. As shown in FIG. 5, and similar to the pulse oximetry system 10 of FIG. 3 and FIG. 4, the pulse oximetry system 10 of FIG. 5 includes the sensor 12, the display 20, and/or the speaker 22, among other components not explicitly listed herein. Differing from the pulse oximetry system 10 of FIG. 3 and FIG. 4, the pulse oximetry system 10 of FIG. 5 (hereinafter referred to as the “pulse oximeter 10”) may wirelessly interact with the client devices 102-106 via the wireless network 110.

The pulse oximeter 10 may additionally include an external data processing unit (not depicted). For example, a CPU (not depicted), incorporated within the pulse oximeter 10, may transmit the physiological parameters to the external data processing unit. Moreover, in some examples, the external data processing unit may be cellular enabled and, in some examples, may incorporate SMC cellular patented technology, among other technologies not explicitly listed herein.

Specifically, in an embodiment, the external data processing unit may be comprised of a cellular modem (not depicted), wherein said modem is able to communicate and/or transmit the physiological parameters to one or more of the client devices 102-106. It should be appreciated that, as described herein, the cellular modem is a device that adds cellular connectivity to devices such as, laptops, desktop computers, tablets, etc. Furthermore, it should be appreciated that the cellular modem may replace existing BLE modules in Bluetooth enabled devices as described herein.

In a further embodiment, the cellular modem may be embedded within the external data processing unit and/or a standalone device connected to the external data processing unit. The connection between the external data processing unit and the cellular modem may be achieved via, a USB connection. As a nonlimiting example, the cellular modem may be selected from the group consisting of AT&T Momentum, Verizon 551 L, USB cellular modems, and motherboard mounted cellular chipsets manufactured by Novatel Wireless, Sierra Wireless, Huawei, and the like. In a further nonlimiting example, the cellular modem may operate by switching between cellular and satellite communications.

Furthermore, the cellular modem may be configured to automatically connect to a slower network when the faster network is not available. The cellular modem may also monitor the reliability of all available connections. The reliability of a network (e.g., the wireless network 110) may be determined from information collected by the cellular modem, which includes, but is not limited to, signal strength, quality, availability, packet loss, retransmits, packet latency, throughput speed, and other cell tower signaling quality factors. The cellular modem may then compare the aforementioned information to a reliability threshold for determining whether to maintain or terminate a connection to the network. The reliability threshold is often automatically set by a cellular carrier or may be manually set by the user of the external data processing unit.

Further, it should be appreciated that the cellular modem is also configured to establish a connection with cellular networks in which the cellular modem is located. The cellular modem may be configured to monitor and detect all cellular networks, comprising the wireless network 110, as the cellular modem moves from one network coverage area to another network coverage area. The cellular modem may detect when a connection to the wireless network 110 is made. For example, the cellular modem may detect whether the wireless network 110 is a 3G, 4G, or 5G network, as well as which cellular network provider (e.g., AT&T, T-Mobile, Verizon, etc.) the modem has connected to.

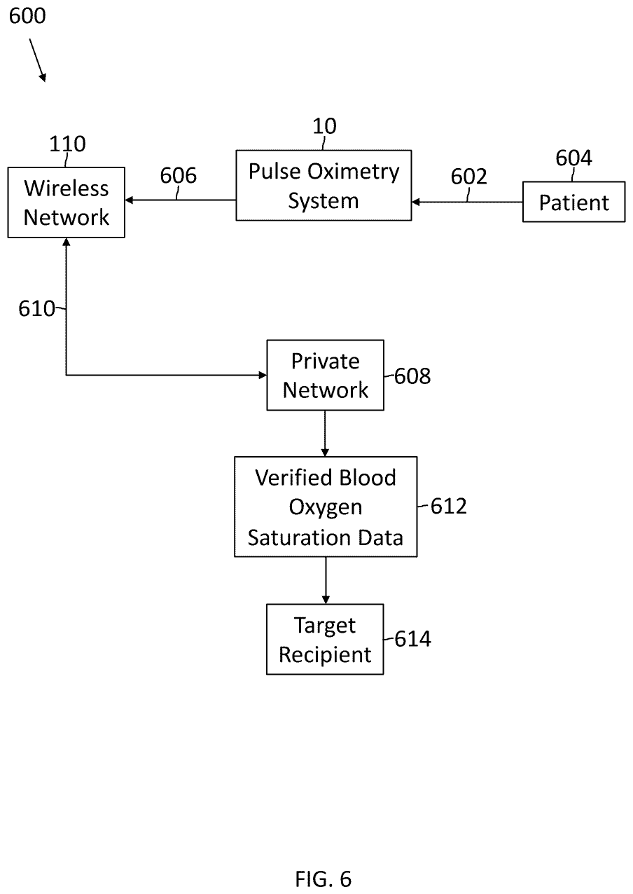

Referring to FIG. 6, the systems and methods of layering security for cellular-enabled blood oxygen saturation data transmission (the “system”) 600 may include the pulse oximeter 10.

In an embodiment, the pulse oximeter 10 collects initial blood oxygen saturation data 602 from the patient 604. Further, the pulse oximeter 10 may utilize various transmission protocol means, such as, but not limited to USSD message transmission technology, CMDA, SMS, GSM, and/or GPRS technology. Through said transmission protocols, at least one of messages and the initial blood oxygen saturation data 602 may be transmitted to a central database where said messages and data 602 are stored.

Upon collection of the initial blood oxygen saturation data 602, the pulse oximeter 10 may encrypt the initial blood oxygen saturation data 602, thus transforming said data 602 into encrypted blood oxygen saturation data 606. In an embodiment, the pulse oximeter 10 may encrypt the initial blood oxygen saturation data 602 with a shared secret.

In one embodiment, the shared secret may consist of a specific piece of data, such as a Personal Identification Number (PIN) or password. The shared secret may enable two or more parties to securely exchange information. Specifically, after encrypted information is exchanged, the shared secret may enable the parties to decrypt the information, ensuring that only those with access to the shared secret can access the content.

In another embodiment, the shared secret may be shared prior to transmission of the encrypted blood oxygen saturation data 606 and/or created at the start of transmission of the data 606. In a nonlimiting example, if the shared secret is shared prior to the transmission, the shared secret may be referred to as a pre-shared key. As a further nonlimiting example, the shared secret, may be created at the start of the transmission with a key-agreement protocol. In yet a further nonlimiting example, the shared secret may be at least one of an asymmetric-key algorithm and a symmetric-key algorithm.

In an embodiment, the symmetric-key algorithm may utilize a key to convert the initial blood oxygen saturation data 602 into the encrypted blood oxygen saturation data 606. In a nonlimiting example, the symmetric-key algorithm may be comprised of at least one of a key and a symmetric block cipher. In a further embodiment, the key may be at least one of a 128-bit key, a 256-bit key, a 576-bit key, and a 2040-bit key. However, any suitable size bit key alternative may comprise the key. In yet another embodiment, the symmetric block cipher may be comprised of at least one of an Advanced Encryption Standard (AES) block cipher, a Blowfish block cipher, a CAST-256 block cipher, a GOST block cipher, an International Data Encryption Algorithm (IDEA) block cipher, a Rivest Cipher 6 (RC-6) block cipher, a Serpent block cipher, and a Twofish block cipher. However, any suitable symmetric block cipher alternative may be utilized.

Additionally, upon creation of the encrypted blood oxygen saturation data 606, the pulse oximeter 10 may sign said data 606 via a signing algorithm, thus creating a data signature. For example, the encrypted blood oxygen saturation data 606 may be cryptographically signed. The encrypted blood oxygen saturation data 606 may be signed via the signing algorithm, which may include at least one of Rivest-Shamir-Adleman (RSA) algorithms, EIGamal signature scheme, Digital Signing Algorithm (DSA), and Elliptical Curve Digital Signature Algorithm (ECDSA). For example, the signing algorithm generates a first hash to accompany the encrypted blood oxygen saturation data 606.

Further, the pulse oximeter 10 may connect to the wireless network 110. In an embodiment, the pulse oximeter 10 may connect to the wireless network 110 via the external data processing unit. For example, the cellular modem, embedded within the external data processing unit, may connect to the wireless network 110. In another example, the cellular modem may connect to the external data processing unit via a USB cable. Such a connection to the wireless network 110 may be achieved via an Access Point Name (APN). As a nonlimiting example, the APN may be a private APN. In an additional embodiment, the APN may require the client devices 102-106 and/or the pulse oximeter 10 to be authorized prior to accessing the wireless network 110. The authorization may register the client devices 102-106 and/or the pulse oximeter 10 via a computing device identifier. The computing device identifier may be at least one of a Subscriber Identification Module (SIM), an International Mobile Equipment Identity (IMEI), and an Integrated Circuit Card Identification Number (IICID).

After the pulse oximeter 10 connects to the wireless network 110, the encrypted blood oxygen saturation data 606 may be transmitted. For example, the encrypted blood oxygen saturation data 606 may be transmitted to a private network 608. In an embodiment, the encrypted blood oxygen saturation data 606 may be transmitted from the wireless network 110 to the private network 608 via a tunnel 610. For example, the tunnel 610 may connect the wireless network 110 to the private network 608, such that the encrypted blood oxygen saturation data 606 may travel from the wireless network 110 to the private network 608, or vice versa. As a nonlimiting example, the tunnel 610 may be a persistent and fully redundant Internet Protocol Security (IPsec) Virtual Private Network (VPN) tunnel. Moreover, the tunnel 610 may leverage the symmetric-key algorithm to encrypt and protect the encrypted blood oxygen saturation data 606 while traveling through the tunnel 610. In another embodiment, the tunnel 610 may also utilize Transport Layer Security (TLS) as another form of protection for transmitting the encrypted blood oxygen saturation data 606 through the tunnel 610.

Further, once the encrypted blood oxygen saturation data 606 has travelled through the tunnel 610, said data 606 may be received by the private network 608. In an embodiment, the system 600 may generate an acknowledgment that is sent to the pulse oximetry system 10 upon acceptance of the encrypted blood oxygen saturation data 606 by the private network 608.

Upon receipt of the encrypted blood oxygen saturation data 606, the private network 608 may verify the data signature of said data 606. For example, the private network 608 may compute a second hash at ingest of the encrypted blood oxygen saturation data 606. Moreover, the second hash may be compared with the first hash. If said first and second hash are a match, then the private network 608 may accept the encrypted blood oxygen saturation data 606, thus verifying the authenticity of said data 606. If the first and second hash are not a match the private network 608 may reject the encrypted blood oxygen saturation data 606, thus ensuring the data 606 comes from a verified source.

Additionally, the private network 608 may decrypt the encrypted blood oxygen saturation data 606 after verifying the first hash and the second hash are a match, thus transforming said data 606 into verified blood oxygen saturation data 612. The verified blood oxygen saturation data 612 may then be quality controlled and/or stored. Further, the verified blood oxygen saturation data 612 may be transmitted to a target recipient 614. In such an embodiment, the verified blood oxygen saturation data 612 may be transmitted to one or more of the client devices 102-106 of the target recipient 614. In a further embodiment, the target recipient 614 may be the patient 604 whom the verified blood oxygen saturation data 612 corresponds to. In another embodiment, the target recipient 614 may be a healthcare provider (e.g., a physician, a nurse, etc.) for the patient 604.

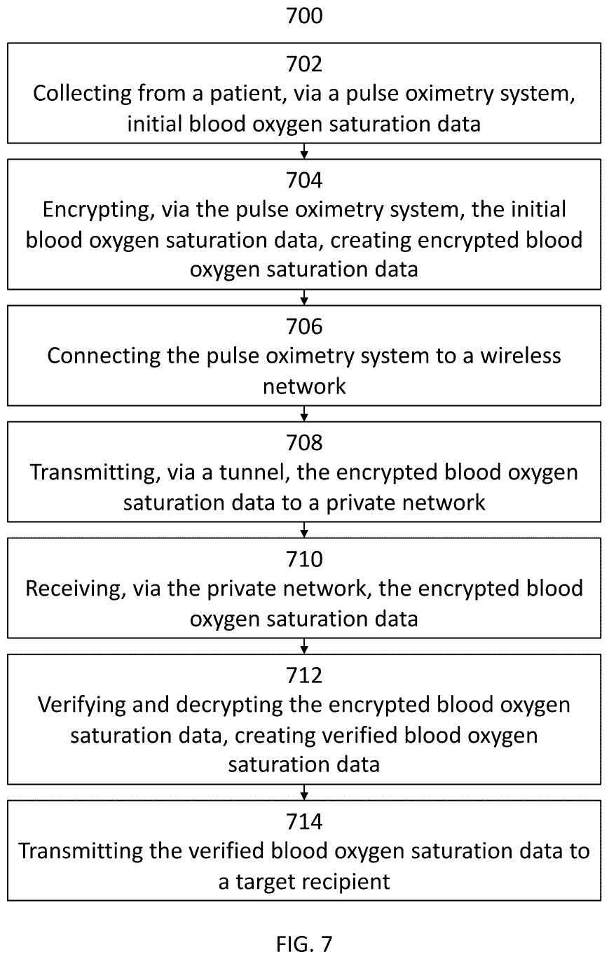

Turning to FIG. 7, a method of layering security for cellular-enabled blood oxygen saturation data transmission (the “method”) 700 may be comprised of at least a first step 702.

In the first step 702, the pulse oximeter 10 may collect the initial blood oxygen saturation data 602 from the patient 604.

In a second step 704 of the method 700, after collecting the initial blood oxygen saturation data 602 from the patient 604, the pulse oximeter 10 may encrypt, and sign said data 602, thus transforming it into encrypted blood oxygen saturation data 606. In an embodiment, the pulse oximeter 10 may encrypt the initial blood oxygen saturation data 602 with the shared secret, wherein the shared secret may be the symmetric-key algorithm. In another embodiment, the symmetric-key algorithm may be comprised of the key and the symmetric block cipher. For example, the symmetric block cipher may be AES-256. Moreover, the encrypted blood oxygen saturation data 606 may be signed via the signing algorithm, wherein the first hash is created.

The method 700 may be further comprised of a third step 706, wherein the pulse oximeter 10 may connect to the wireless network 110. In an embodiment, the connection may be achieved via the APN.

Additionally, a fourth step 708 may be employed, wherein the encrypted blood oxygen saturation data 606 is transmitted to the private network 608 from the pulse oximeter 10 via the tunnel 610. In an embodiment, the encrypted blood oxygen saturation data 606 may first be transmitted from the pulse oximeter 10 to the wireless network 110, and then from the wireless network 110 to the private network 608 via the tunnel 610. In another embodiment, the tunnel 610 may be a persistent and fully redundant IPsec VPN tunnel. Furthermore, the tunnel 610 may also leverage TLS, as an additional form of protection for transmitting the encrypted blood oxygen saturation data 606 through the tunnel 610.

A fifth step 710 of the method 700 may entail the private network 608 receiving the encrypted blood oxygen saturation data 606. In an embodiment, upon receipt of the encrypted blood oxygen saturation data 606, the private network 608 may transmit an acknowledgment to the pulse oximeter 10.

Furthermore, the method 700 may employ a sixth step 712, wherein the private network 608 may verify and decrypt the encrypted blood oxygen saturation data 606. The verification and decryption of the encrypted blood oxygen saturation data 606 may transform said data 606 into verified blood oxygen saturation data 612. In such a step 712, the second hash may be generated upon receipt of the encrypted blood oxygen saturation data 606, wherein said second hash is then compared to the first hash. Such a comparison may act as a verification of the source of encrypted blood oxygen saturation data 606.

The method 700 may further include a seventh step 714, wherein the verified blood oxygen saturation data 612 is quality controlled and/or relayed to the target recipient 614. In an embodiment, the target recipient 614 may be the patient 604 whom the verified blood oxygen saturation data 612 corresponds to and/or a healthcare provider (e.g., a physician, a nurse, etc.) for the patient 604.

As described herein, “NFC” is a set of communication protocols for communication between two electronic devices over a distance of 4 cm or less. NFC devices can act as electronic identity documents and keycards and may be used in contactless payment systems and allow mobile payment replacing or supplementing systems such as credit cards and electronic ticket smart cards. NFC can be used for sharing small files such as contacts and bootstrapping fast connections to share larger media such as photos, videos, and other files.

In an embodiment, at least one of the system 600 and the method 700 may aid in the prevention of a data breach via a cyberattack. For example, layering two or more of: (1) encrypting the initial blood oxygen saturation data 602; (2) connecting the pulse oximeter 10 to the wireless network 110 via the APN; (3) transmitting the encrypted blood oxygen saturation data 606 from the wireless network 110 to the private network 608 via the tunnel 610; (4) generating the acknowledgement and sending it to the pulse oximeter 10 upon the private network's 608 acceptance of the encrypted blood oxygen saturation data 606; (5) verifying the data signature of the encrypted blood oxygen saturation data 606 and decrypting said data 606; and (6) enabling the target recipient 614 to authenticate the sender of the verified blood oxygen saturation data 612 may better safeguard remote data transmissions of protected healthcare information from cellular-enabled devices than currently existing systems and methods. As a nonlimiting example, layering 1, 2, and 3 above ensures that layer 2 reinforces layer 1 and that layer 3 reinforces layer 2. The redundancy in layering security measures creates a tamper proof system for transmitting protected healthcare information. Moreover, the industry at large utilizes the public Internet to transmit information without providing origin authentication. However, both the system 600 and method 700 are able to guarantee the origin and authenticity of protected healthcare information by sending encrypted healthcare information through the tunnel 610 from the wireless network 110 to the private network 608 and requiring a comparison and match of the first and second hashes. The aforementioned layering ensures protected healthcare information (i.e., the initial data 602, encrypted data 606, and verified blood oxygen saturation data 612) reaches the target recipient 614, while simultaneously proscribing bad actors from accessing said protected information.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A system for improving security of cellular-enabled blood oxygen saturation data transmission by layering security, the system comprising:
a pulse oximeter;
a wireless network connected to the pulse oximeter;
a private network connected to the wireless network via a persistent and fully redundant Internet Protocol Security (IPsec) Virtual Private Network (VPN) tunnel;
one or more computer processors; and
a memory having stored therein machine executable instructions, that when executed by the one or more processors, cause the system to:
collect, via the pulse oximeter, blood oxygen saturation data from a patient;
encrypt, via the pulse oximeter, the blood oxygen saturation data with a shared secret,
wherein encrypting the blood oxygen saturation data creates encrypted blood oxygen saturation data;
generate, via the pulse oximeter, a first hash using a signing algorithm;
transmit, via the persistent and fully redundant IPsec VPN tunnel, the encrypted blood oxygen saturation data from the pulse oximeter to the private network;
generate, via the private network, a second hash;
compare, via the one or more computer processors, the first hash to the second hash;
decrypt, via the one or more computer processors, the encrypted blood oxygen saturation data upon a match of the first and second hash,
wherein decrypting the encrypted blood oxygen saturation data creates verified blood oxygen saturation data; and
transmit, via the one or more computer processors, the verified blood oxygen saturation data to a target recipient.

2. The system of claim 1, wherein the shared secret is a symmetric-key algorithm comprising:
a key; and
a symmetric block cipher.

3. The system of claim 2, wherein the key is comprised of at least one of a 128-bit key, a 256-bit key, a 576-bit key, and a 2040-bit key.

4. The system of claim 2, wherein the symmetric block cipher is comprised of at least one of an Advanced Encryption Standard (AES) block cipher, a Blowfish block cipher, a CAST-256 block cipher, a GOST block cipher, an International Data Encryption Algorithm (IDEA) block cipher, a Rivest Cipher 6 (RC-6) block cipher, a Serpent block cipher, and a Twofish block cipher.

5. The system of claim 2, wherein the persistent and fully redundant IPsec VPN tunnel leverages the symmetric-key algorithm to encrypt the encrypted blood oxygen saturation data while travelling through the persistent and fully redundant IPsec VPN tunnel.

6. The system of claim 1, wherein the pulse oximeter connects to the wireless network via an Access Point Name (APN).

7. The system of claim 1, wherein the persistent and fully redundant IPsec VPN tunnel is further comprised of Transport Layer Security (TLS).

8. The system of claim 1, wherein the verified blood oxygen saturation data is transmitted to one or more client devices of the target recipient.

9. The system of claim 1, wherein the signing algorithm is comprised of at least one of Rivest-Shamir-Adleman (RSA) algorithms, EIGamal signature scheme, Digital Signing Algorithm (DSA), and Elliptical Curve Digital Signature Algorithm (ECDSA).

10. A method for improving security of cellular-enabled blood oxygen saturation data transmission by layering security, the method comprising:
collecting, via a pulse oximeter, blood oxygen saturation data from a patient;
encrypting, via a shared secret generated by the pulse oximeter, the blood oxygen saturation data,
wherein encrypting the blood oxygen saturation data creates encrypted blood oxygen saturation data;
signing, via a signing algorithm, the encrypted blood oxygen saturation data creating a first hash;
connecting, via an Access Point Name (APN), the pulse oximeter to a wireless network,
transmitting, via a persistent and fully redundant Internet Protocol Security (IPsec) Virtual Private Network (VPN) tunnel, the encrypted blood oxygen saturation data from the pulse oximeter to a private network;
receiving, via the private network, the encrypted blood oxygen saturation data,
wherein upon receipt of the encrypted blood oxygen saturation data, the private network generates a second hash;
verifying, via a comparison of the first hash and second hash, the encrypted blood oxygen saturation data,
wherein upon a match of the first hash and the second hash, the private network decrypts the encrypted blood oxygen saturation data, creating verified blood oxygen saturation data; and
transmitting the verified blood oxygen saturation data to a target recipient.

11. The method of claim 10, wherein the shared secret is a symmetric-key algorithm comprising:
a key; and
a symmetric block cipher.

12. The method of claim 11, wherein the key is comprised of at least one of a 128-bit key, a 256-bit key, a 576-bit key, and a 2040-bit key.

13. The method of claim 11, wherein the symmetric block cipher is comprised of at least one of an Advanced Encryption Standard (AES) block cipher, a Blowfish block cipher, a CAST-256 block cipher, a GOST block cipher, an International Data Encryption Algorithm (IDEA) block cipher, a Rivest Cipher 6 (RC-6) block cipher, a Serpent block cipher, and a Twofish block cipher.

14. The method of claim 11, wherein the persistent and fully redundant IPsec VPN tunnel leverages the symmetric-key algorithm to encrypt the encrypted blood oxygen saturation data while travelling through the persistent and fully redundant IPsec VPN tunnel.

15. The method of claim 10, wherein the persistent and fully redundant IPsec VPN tunnel is further comprised of Transport Layer Security (TLS).

16. The method of claim 10, wherein the signing algorithm is comprised of at least one of Rivest-Shamir-Adleman (RSA) algorithms, EIGamal signature scheme, Digital Signing Algorithm (DSA), and Elliptical Curve Digital Signature Algorithm (ECDSA).
